(19) United States
(12) Patent Application Publication
(10) Pub. No.: US 2021/0315669 A1
(43) Pub. Date: Oct. 14, 2021

(54) Orthodontic suite and its manufacturing method

(71) Applicant: Chi-Ching Huang, Taoyuan City (TW)

(72) Inventors: Chi-Ching Huang, Taoyuan City (TW); Tzu-Yun Kao, Taoyuan City (TW)

(21) Appl. No.: 17/083,383

(22) Filed: Oct. 29, 2020

(30) Foreign Application Priority Data
Apr. 14, 2020 (CN) 202020542871.7
Jul. 6, 2020 (CN) 202010641369.6

(51) Int. Cl. A61C 7/28; A61C 7/00

(52) U.S. Cl. CPC A61C 7/282; A61C 7/002

(57) ABSTRACT

A orthodontic suite and its manufacturing method for multistage treating malocclusion or abnormal alignment of the teeth and jaws are suggest. The orthodontic suite has a first stage and a second orthodontic appliances, in which the first orthodontic appliance includes a first stage arch, first stage anchoring troughs and first stage adapting troughs disposed on the first stage arch. The second orthodontic appliance includes a second stage arch, second stage anchoring troughs and second stage adapting troughs disposed on the second stage arch. The positions of second stage anchoring troughs are identical to the first stage anchoring troughs, and the positions of the second stage adapting troughs are shifting or rotating based on the first stage adapting troughs.

### CROSS-REFERENCE TO RELATED APPLICATION

The present application is based upon and claims priority to Chinese Patent Applications No. 202010641369.6 filed on Jul. 6, 2020 and No. 202020542871.7 filed on Apr. 14, 2020, the content of which is hereby incorporated by reference in its entirety.

### TECHNICAL FIELD

The present invention relates to a dental orthodontic appliance, particularly to a dental appliance cable of clinic treatments for multistage treating malocclusion or abnormal alignment of the teeth and jaws, overdevelopment of dental arch, or having function of teeth germination induction, first molar growth anchorage, so as to improve teeth alignment, occlusive relationship, and regenerate alveolar bone morphology, and finally treat mouth breath and raise sleep quality.

### BACKGROUND

According to the World Health Organization (WHO) statistics, seven out of ten people in the world have different level of problems in teeth or dental arch, such as teeth dislocation, malocclusion, crowding of dentition (hyperdontia), chipped tooth, crooked teeth, underbite or overbite, etc. These dental problems not only bring aesthetic issues, but also cause chewing dysfunction, tooth decay, anterior teeth break, or even temporomandibular disorder, periodontal disease, dyslexia, abnormal development of jawbone, etc. As for the causes, some congenital or hereditary factors, such as maxillary protrusion, mandibular retrusion, congenital missing teeth, or polydentosis, can bring the aforementioned dental issues, however, patient's personal nurture habits might do. No matter what the causes are, those problems such as teeth dislocation or malocclusion are able to be treated and improved through clinic orthodontics, so that dental health, functionality and aesthetics are therefore increased.

Please refer to FIG. 1, FIG. 1 is schematic diagram of traditional orthodontic brace utilized in patient's mouth. As shown in FIG. 1, a metal dental brace 8 is set up to malposition teeth 95 neighboring to side of lip 96 of patient mouth. In few clinic cases, some dental brace 8 could be set up to lingual side; however the obvious problems such as cleaning, adaptability, difficulty or even the financial cost are absolutely worse than previous description as shown in FIG. 1. Thus setting up the dental brace 8 to the labial side (i.e. neighboring to lip 96) is better option to most patients. Traditionally, the metal dental brace 8 has a archwire 81 (usually made of steel) to attach several orthodontic brackets 82, and each of the orthodontic brackets 82 is firmly attached to the malposition tooth 95. In this manner, these malposition teeth 95 is able to be moved, rotated and then corrected through the mechanical force of the archwire 81. Therefore these malposition teeth 95 can be gradually adjusted to right position with right azimuth relative to human's dental arches or jaws, so that the expectation of teeth reshape and orthodontics are certainly achieved.

However, there is always mechanical error or tolerance occurred on the archwire 81 and the orthodontic brackets 82 when the dental brace 8 is set up; therefore the malposition teeth 95 could be overshot adjustment if the mechanical fore of the archwire 81 imposes too much, or have poor correction (end up with longer correction time) if the mechanical force of archwire 81 imposes too little. Besides, the dental brace 8 revealed in FIG. 1 is normally inconvenient to brush teeth, particularly for kids. Some cases shows that utilizing dental brace 8 have obviously raised the chance of caries and periodontal diseases, thus that will increase the limitation and lower the feasibility for the kids.

Due to these issues addressed about orthodontics, it is desirably required to relieve the disadvantages of the archwire 81 and allow the patients to brush and clean teeth with ease, as well as have better feasibility and practicability to all ages of patients including adults and kids. It will be clear to those skilled in the art how to have better clinic experience and make the whole process accessible.

### SUMMARY

The primary objective of the present invention is to relieve the disadvantages of the metal archwire of dental brace utilized in orthodontics, and to overcome the limitation for correcting azimuth and movement, so as to have higher accuracy on tooth correction and thus raise controllability.

The other objective of the present invention is to provide a multistage orthodontic appliance, so as to treat the malocclusion, malposition or abnormal alignment of the teeth and jaws, reshape and regenerate jawbone through biological morphology of orthodontics, and even further address the hypoplasia or overdevelopment of dental arch. Furthermore, all of these issues will be taken care with some other considerations such as tooth brushing and cleaning with ease and convenience.

The further other objective of the present invention is to make orthodontic treatment, dental arch adjustment, occlusion adjustment, teeth reshape, and malocclusion treatment adaptable to all ages including adults and kids, as well as teeth germination induction can also be achieved. Thus the dislocated, malalignment teeth or malocclusive jaws are able to be adjusted and moved to proper position so as to meet Centric Relation (CR) and Class I occlusion of Angle's Classification, therefore the stability of occlusion and teeth positions are as a result improved.

The still other objective of the present invention is to prevent respiratory tract obstruction, reduce severe snore and mouth breathing caused from lower resting position of the tongue, so as to have breath training and relieve negative impact of snore and sleep apnea, and finally improve one's sleep quality.

For dealing with those aforementioned issues addressed, the present invention suggests a orthodontic suite for multistage treating malocclusion or abnormal alignment of the teeth and jaws. The present invention of the orthodontic suite comprises a first stage orthodontic appliance and a second stage orthodontic appliance. The first stage orthodontic appliance has a first stage arch, a pluralities of first stage anchoring troughs and a pluralities of first stage adapting troughs, with the first stage anchoring troughs and the first stage adapting troughs disposed on the first stage arch. The first stage anchoring troughs are disposed through shifting sites of patient's first molars along Buccal Side direction BS by a first expanding space d1. The first stage adapting troughs are disposed through shifting sites of patient's other teeth along Buccal Side direction BS by a second expanding space d2. The second stage orthodontic appliance has a second stage arch, a pluralities of second stage anchoring troughs and a pluralities of second stage adapting troughs, with the second stage anchoring troughs and the second stage adapting troughs disposed on the second stage arch. The second stage anchoring troughs are disposed at the same sites with the first stage anchoring troughs. The second stage adapting troughs are disposed through shifting or rotating sites of the first stage adapting troughs.

According to an embodiment of the aforesaid orthodontic suite, wherein the first stage anchoring troughs or second stage anchoring troughs meet Class I occlusion relationship of Angle's Classification.

According to an embodiment of the aforesaid orthodontic suite, wherein the second expanding space d2 is smaller than or equal to 1.2 times the first expanding space d1, and larger than 0.2 times the first expanding space d1.

According to an embodiment of the aforesaid orthodontic suite, wherein the second stage adapting troughs are disposed through shifting the sites of the first stage adapting troughs by one fourth to one third of tooth's section width, or rotating the sites of the first stage adapting troughs by an angle less than 30 degree.

According to an embodiment of the aforesaid orthodontic suite, further having a third stage orthodontic appliance, wherein the third stage orthodontic appliance has a third stage arch, a pluralities of third stage anchoring troughs and a pluralities of third stage adapting troughs, with the third stage anchoring troughs and the third stage adapting troughs disposed on the third stage arch. The third stage anchoring troughs are disposed at the same sites with the second stage anchoring troughs, and the third stage adapting troughs are disposed through shifting or rotating sites of the second stage adapting troughs. In the further specific embodiments, the third stage adapting troughs are disposed through shifting the sites of the second stage adapting troughs by one fourth to one third of tooth's section width, or rotating the sites of the second stage adapting troughs by an angle less than 30 degree.

According to an embodiment of the aforesaid orthodontic suite, further having a first stage tongue support and a second stage tongue support, with the first stage tongue support disposed at inner side of the first stage arch and the second stage tongue support disposed at inner side of the second stage arch. In the further specific embodiments, the first stage tongue support or the second stage tongue support gradually slopes downward along the opposite of Labial Side direction LS.

According to an embodiment of the aforesaid orthodontic suite, further having a first stage tongue support and a second stage tongue support, with the first stage tongue support disposed at inner side of the first stage arch and the second stage tongue support disposed at inner side of the second stage arch. In the further specific embodiments, the first stage tongue support has a slot disposed on midline of the first stage arch, or the second stage tongue support has a slot disposed on midline of the second stage arch.

For dealing with those aforementioned issues addressed, the present invention suggests a manufacturing method of orthodontic suite. The present invention of manufacturing method of orthodontic suite comprises the following steps: Step 11: obtaining patient's dental images; Step 12: setting a first expanding space d1 from the patient's first molar toward patient's cheek along Buccal Side direction BS; Step 13: arranging a first stage anchoring trough through shifting position of the first molar by the first expanding space d1; Step 14: setting a pluralities of second expanding spaces d2 on other teeth toward the Buccal Side direction BS based on relative positions with the first stage anchoring trough; Step 15: arranging a pluralities of first stage adapting troughs on the distal of the second expanding spaces d2 according to category of the teeth; Step 16: arranging the first stage anchoring trough and the pluralities of first stage adapting troughs on a quadrant of first stage arch to form a 3D digital profile of a first stage orthodontic appliance; Step 17: obtaining a physical model of the first stage orthodontic appliance; Step 21: obtaining patient's dental images after first stage treatment; Step 22: arranging a second stage anchoring trough based on position of the first stage anchoring trough; Step 23: arranging and adjusting a pluralities of second stage adapting troughs based on the first stage adapting troughs; Step 24: arranging the second stage anchoring trough and the pluralities of second stage adapting troughs on a quadrant of second stage arch, to form a 3D digital profile of a second stage orthodontic appliance; Step 25: obtaining a physical model of the second stage orthodontic appliance.

According to an embodiment of the aforesaid manufacturing method of orthodontic suite, wherein the first stage anchoring trough or second stage anchoring trough meets Class I occlusion relationship of Angle's Classification.

According to an embodiment of the aforesaid manufacturing method of orthodontic suite, wherein the second expanding space d2 of patient's incisor in Step 14 is smaller than or equal to 0.4 times the first expanding space d1, and larger than 0.2 times the first expanding space d1.

According to an embodiment of the aforesaid manufacturing method of orthodontic suite, wherein the second expanding space d2 of patient's canine in Step 14 is smaller than or equal to 0.8 times the first expanding space d1, and larger than 0.4 times the first expanding space d1.

According to an embodiment of the aforesaid manufacturing method of orthodontic suite, wherein the second expanding space d2 of patient's other molars in Step 14 are smaller than or equal to 1.2 times the first expanding space d1, and larger than 0.8 times the first expanding space d1.

According to an embodiment of the aforesaid manufacturing method of orthodontic suite, wherein the second stage adapting troughs in the Step 23 are arranged through shifting the positions of the first stage adapting troughs by one fourth to one third of tooth's section width.

According to an embodiment of the aforesaid manufacturing method of orthodontic suite, wherein the second stage adapting troughs in the Step 23 are arranged through rotating the sites of the first stage adapting troughs by an angle less than 30 degree.

According to an embodiment of the aforesaid manufacturing method of orthodontic suite, further comprising the following steps: Step 31: obtaining patient's dental images after second stage treatment; Step 32: arranging a third stage anchoring trough based on position of the second stage anchoring trough; Step 33: arranging and adjusting a pluralities of third stage adapting troughs based on the second stage adapting troughs; Step 34: arranging the third stage anchoring trough and the pluralities of third stage adapting troughs on a quadrant of third stage arch, to form a 3D digital profile of a third stage orthodontic appliance; Step 35: obtaining a physical model of the third stage orthodontic appliance. In the further specific embodiments, the third stage orthodontic appliance is composed of a higher part arch and a under part arch, with the higher part arch and the under part arch separately fabricated.

According to an embodiment of the aforesaid manufacturing method of orthodontic suite, wherein the first stage orthodontic appliance or the second stage orthodontic appliance is composed of a higher part arch and a under part arch, with the higher part arch and the under part arch separately fabricated.

Summarily, the present invention of orthodontic suite can relieve the disadvantages of the metal archwire of dental brace utilized in orthodontics, and to overcome the limitation for correcting azimuth and movement, so as to have higher accuracy on tooth correction and thus raise controllability. Secondly, the present invention is able to reshape and regenerate jawbone through biological morphology of orthodontics, and even address the hypoplasia or overdevelopment of dental arch. Thirdly, the orthodontic suite can allow the patients to brush and clean teeth with ease, as well as have better feasibility and practicability to all ages of patients including adults and kids, and simultaneously treat the malocclusion, malposition or abnormal alignment of the teeth and jaws. Fourthly the orthodontic suite can still more achieve teeth germination induction, and adjust and move the dislocated, malalignment teeth or malocclusive jaws to proper position, so as to meet Centric Relation (CR) and Class I occlusion of Angle's Classification, and therefore improve the stability of occlusion and teeth positions. Thus the present invention have huge potential in both clinic practice and commercialization.

For further understanding of the present invention, reference is made to the following detailed description illustrating the embodiments and examples of the present invention. The description is for illustrative purpose only and is not intended to limit the scope of the claim.

### BRIEF DESCRIPTION OF THE DRAWINGS

The accompanying drawings, which are incorporated in and constitute a part of this specification, illustrate embodiments consistent with the invention and, together with the description, serve to explain the principles of the present disclosure.

FIG. 1 is schematic diagram of traditional orthodontic brace utilized in patient's mouth.

FIG. 2 is flow chart showing multistage manufacturing method of orthodontic suite.

FIG. 3 is flow chart showing the manufacturing method of first stage orthodontic appliance.

FIG. 4 is schematic diagram of patient's image of dental arch.

FIG. 5A˜5C are schematic diagrams showing manufacture procedure of the first stage orthodontic appliance.

FIG. 6A˜6B are schematic diagrams showing perspective view and top view of the first stage orthodontic appliance.

FIG. 7A˜7C are schematic diagrams showing dental arch and the first stage orthodontic appliance before and after the patient's occlusion.

FIG. 8 is schematic diagram showing structural relationship between patient's teeth and troughs of the first stage orthodontic appliance.

FIG. 9 is flow chart showing the manufacturing method of second stage orthodontic appliance.

FIG. 10 is schematic diagram showing structural relationship between troughs of the first stage orthodontic appliance and the second stage orthodontic appliance.

FIG. 11 is flow chart showing the manufacturing method of third stage orthodontic appliance.

FIG. 12 is schematic diagram showing the orthodontic appliance with the higher part arch and the under part arch separately fabricated.

FIG. 13A˜13B are schematic diagrams showing higher part arch and under part arch of the orthodontic appliance are made in different stage.

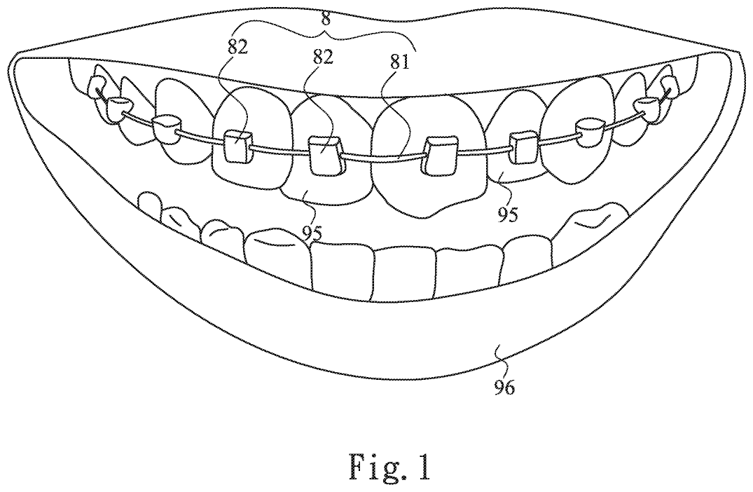

### DETAILED DESCRIPTION

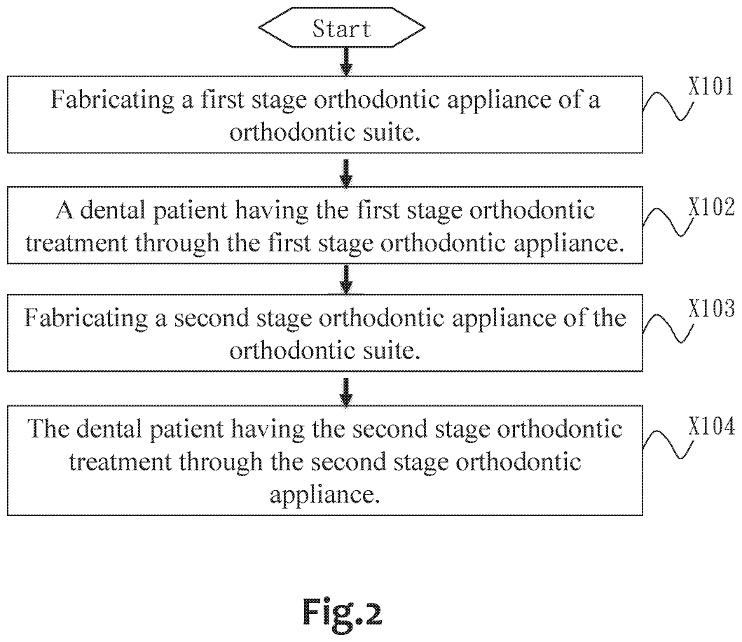

Dental treatment of orthodontics is specific practice for oral cavity, which can have function such as teeth correction, teeth reshape or malocclusion treatment, so as to achieve perfect teeth alignment and beautiful profile of dental arch. In order to achieve that, the present invention suggests a orthodontic suite and a manufacturing method to produce the orthodontic suite. Please refer to FIG. 2, FIG. 2 is flow chart showing multistage manufacturing method of orthodontic suite. As shown in FIG. 2, a manufacturing method of orthodontic suite 10 is provided, which firstly fabricates a first stage orthodontic appliance 10 of a orthodontic suite (Step X101), so that a dental patient may have the first stage orthodontic treatment through the first stage orthodontic appliance 10 (Step X102). According to the clinic outcome after the first stage treatment, then it shall fabricate a second stage orthodontic appliance 20 of the orthodontic suite (Step X103), so that the dental patient may have the second stage orthodontic treatment through the second stage orthodontic appliance 20 (Step X104).

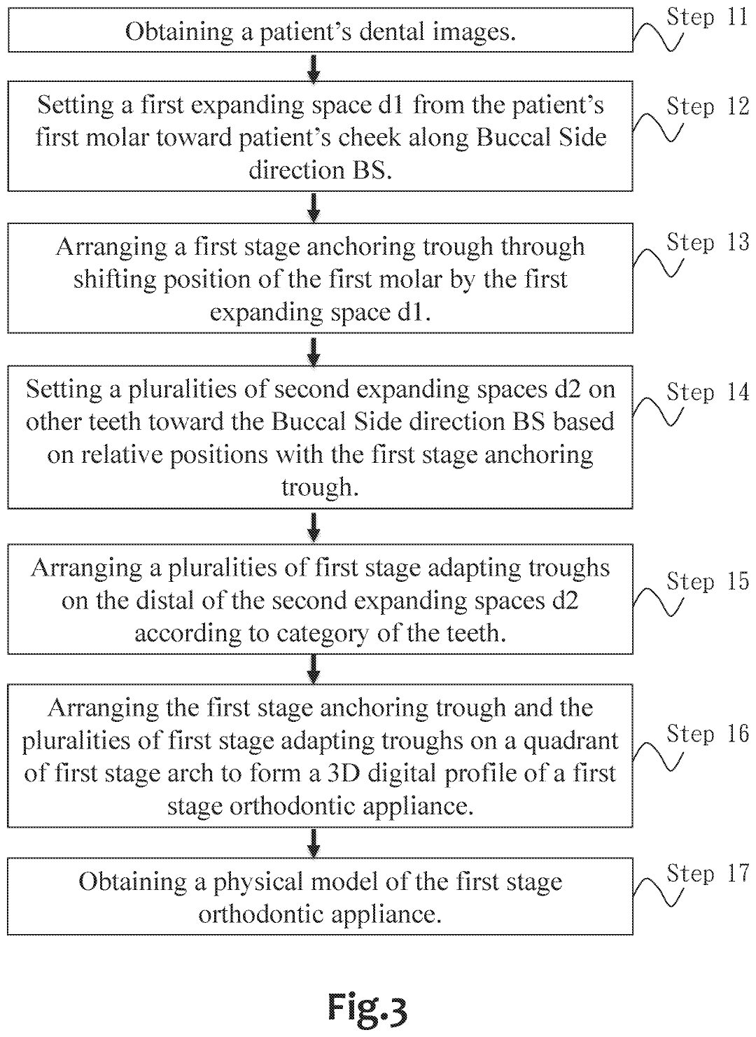

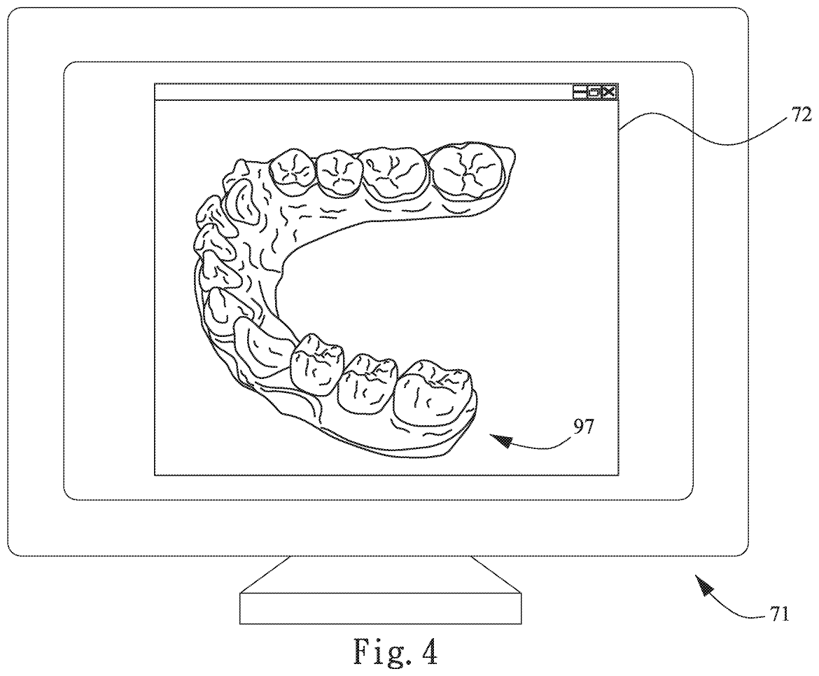

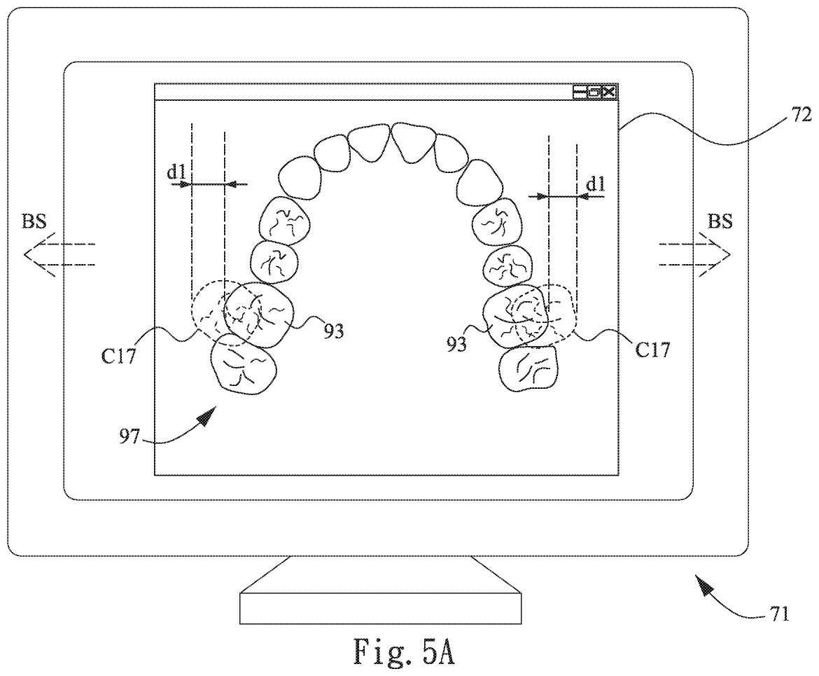

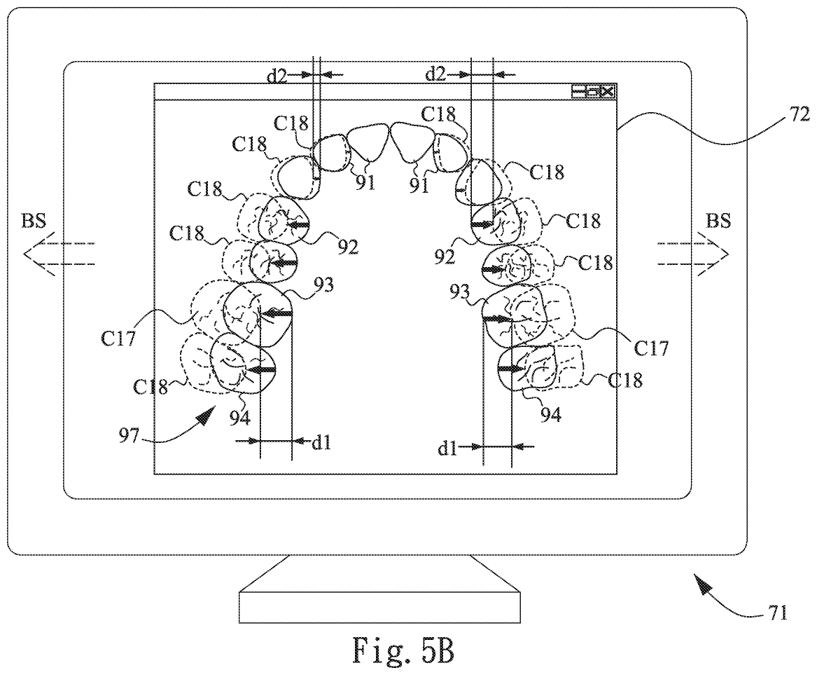

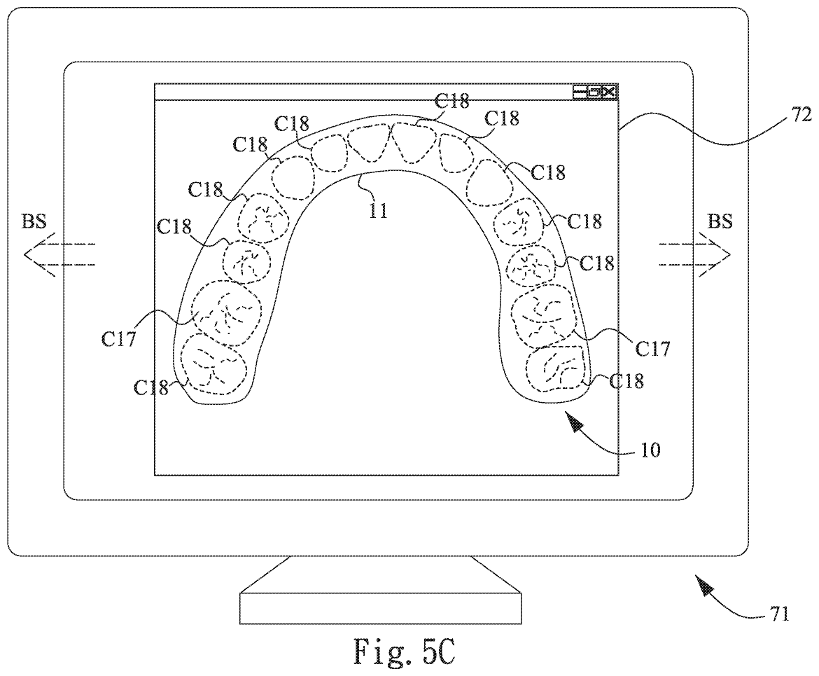

Please refer to FIG. 3, FIG. 3 is flow chart showing the manufacturing method of first stage orthodontic appliance. Specifically, the Step X101 can have further sub-steps, and be broken down into Steps 11˜17 as demonstrated in FIG. 3. As shown in FIG. 3, a patient's dental images can be obtained (Step 11) through Computed Tomography (CT), X-Ray, nuclear magnetic resonance or ultrasonic instruments, or oral scan or dental arch molding technique. These dental images includes but not limits in DICOM-format (Digital Imaging and Communications in Medicine) photos or other digital pictures. As show in FIG. 4, the aforementioned dental images can be obtained and recorded through a computer 71 (including and not limiting on smartphone, server or other hardware devices) or a dental software 72, so that 3D profile, structural relationship and visualization features of a dentition 97 or dental arch are thus achieved. In this manner, the location, orientation, azimuth or surficial profile of each tooth before orthodontic treatment is therefore obtained. Then, a Buccal Side direction BS going from interior toward cheek of the patient is defined on the dentition 97 and the dental arch. As shown in FIG. 5A, a first expanding space d1 is set from the patient's first molar 93 toward patient's cheek along Buccal Side direction BS (Step 12 within FIG. 3), and arrange a first stage anchoring trough C17 through shifting position of the first molar 93 by the first expanding space d1 (Step 13). In this manner, the location of the first stage anchoring trough C17 is exactly the site which the dental patient's first molar 93 is planned to move to, so that the first stage anchoring trough C17 meets Class I occlusion relationship of Angle's Classification. Wherein the Angle's Classification classifies malocclusion based on the relative position of the maxillary first molar 93 within the dentition 97. The Angle's Classification classifies occlusion into 3 types; firstly, Class I: Neutrocclusion. Class I of Angle's Classification means normal occlusion, in which the incisor of upper jaw covers and is anterior to the incisor of the lower jaw by 1 mm to 3 mm. Secondly, Class II: Distocclusion. Class II of Angle's Classification means the incisor of upper jaw is too much anterior to the incisor of lower jaw, namely excess overjet or overbite, or compensatory retraction of maxillary incisor leading to skeletal buck teeth. Thirdly, Class III: Mesiocclusion. Class III of Angle's Classification means the incisor of lower jaw is anterior to the incisor of upper jaw, so as to become negative overjet or anterior crossbite, or have symptoms of maxillary retraction or mandible protrusion (so-called underbite).

Afterward, as shown in FIG. 5B, a pluralities of second expanding spaces d2 are set on other teeth toward the Buccal Side direction BS, based on relative positions with the first stage anchoring trough C17 (Step 14 within FIG. 3); wherein the “other teeth” means all teeth includes incisors 91, canines 92 and other molars 94, excluding the first molar 93. In practice, each second expanding space d2 of all teeth will be unequal, therefore dentist or dental technician needs to decide the value of the second expanding space d2 according to age of the patient or future growth of patient's dental arch; since kid is expected to have huge expanding potentials on his/her dental arch. Clinically the second expanding space d2 is smaller than or equal to 1.2 times the first expanding space d1, and larger than or equal to 0.2 times the first expanding space d1; namely 0.2*d1≤d2≤1.2*d1. Specifically, the second expanding space d2 of patient's incisor 91 is smaller than or equal to 0.4 times the first expanding space d1, and larger than or equal to 0.2 times the first expanding space d1; namely 0.2*d1≤d2≤0.4*d1. Further, the second expanding space d2 of patient's canine 92 is smaller than or equal to 0.8 times the first expanding space d1, and larger than or equal to 0.4 times the first expanding space d1; namely 0.4*d1≤d2≤0.8*d1. Moreover, the second expanding spaces d2 of patient's other molars 94 are smaller than or equal to 1.2 times the first expanding space d1, and larger than or equal to 0.8 times the first expanding space d1; namely 0.8*d1≤d2≤1.2*d1. Therefore the dentist or dental technician can arrange a pluralities of first stage adapting troughs C18 on the distal of the second expanding spaces d2 according to category of the teeth (Step 15 within FIG. 3), and then the first stage adapting troughs C18 are able to correspond to all teeth other than the first molars 93.

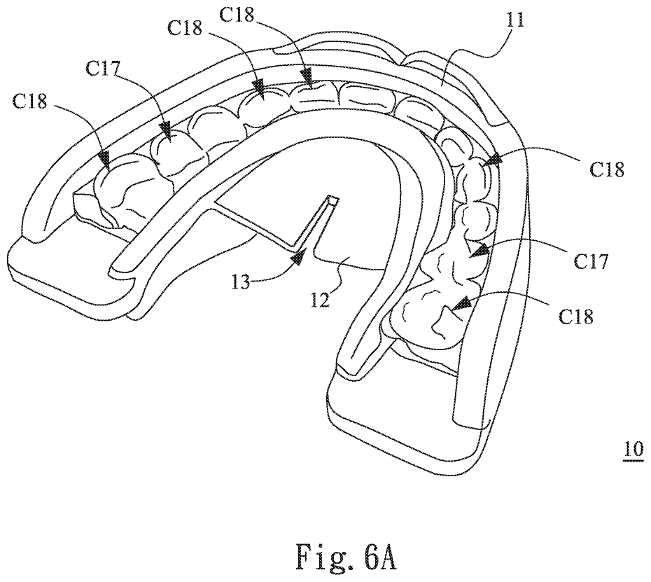

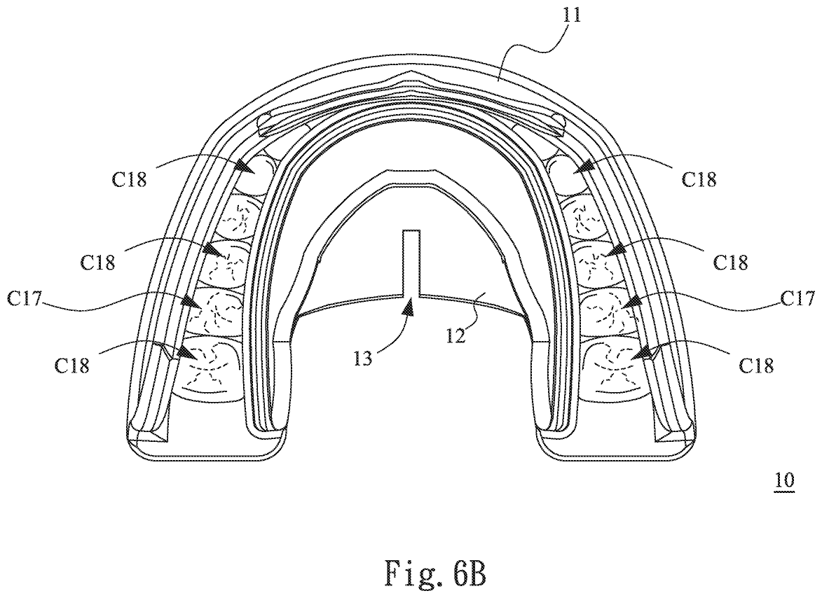

As shown in FIG. 5C, the first stage anchoring trough C17 and the pluralities of first stage adapting troughs C18 are arranged on a quadrant of first stage arch 11, so as to form a 3D digital profile of a first stage orthodontic appliance 10 (Step 16 within FIG. 3), wherein the first stage arch 11 comprises right-side quadrant and left-side quadrant. Afterward, a physical model of the first stage orthodontic appliance 10 (Step 17 within FIG. 3) is hence obtained. In the preferred embodiment, the physical model of the first stage orthodontic appliance 10 is made through 3D Printing, and demonstrated in FIG. 6A and FIG. 6B.

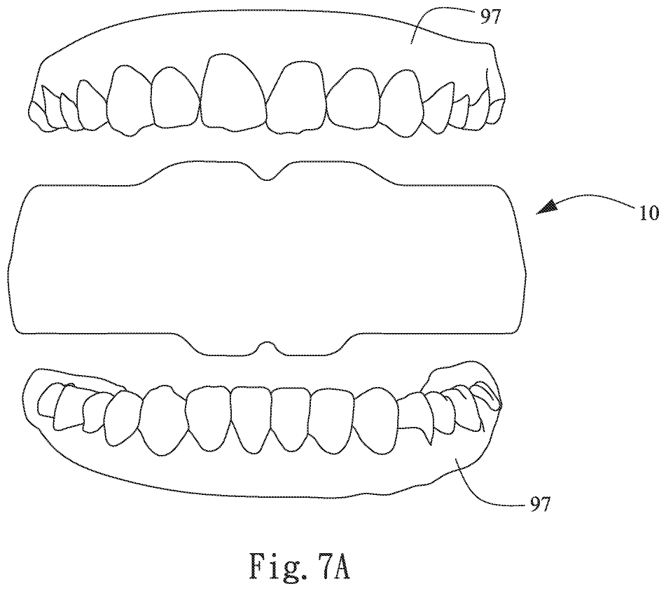

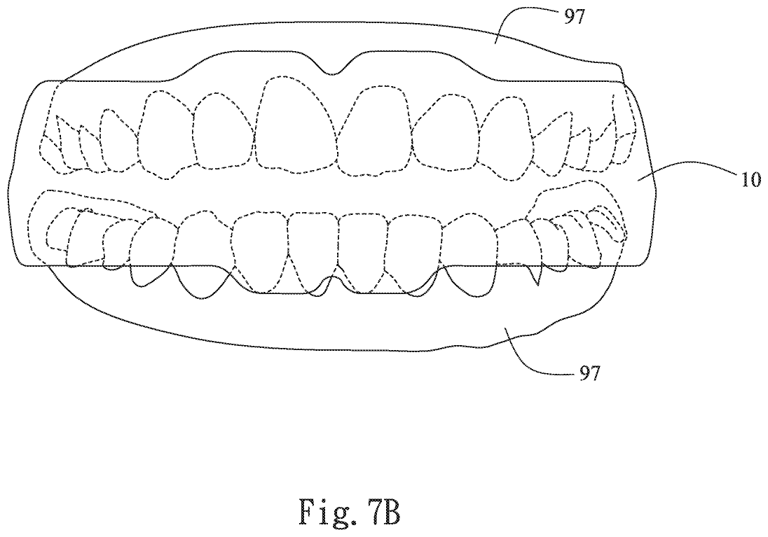

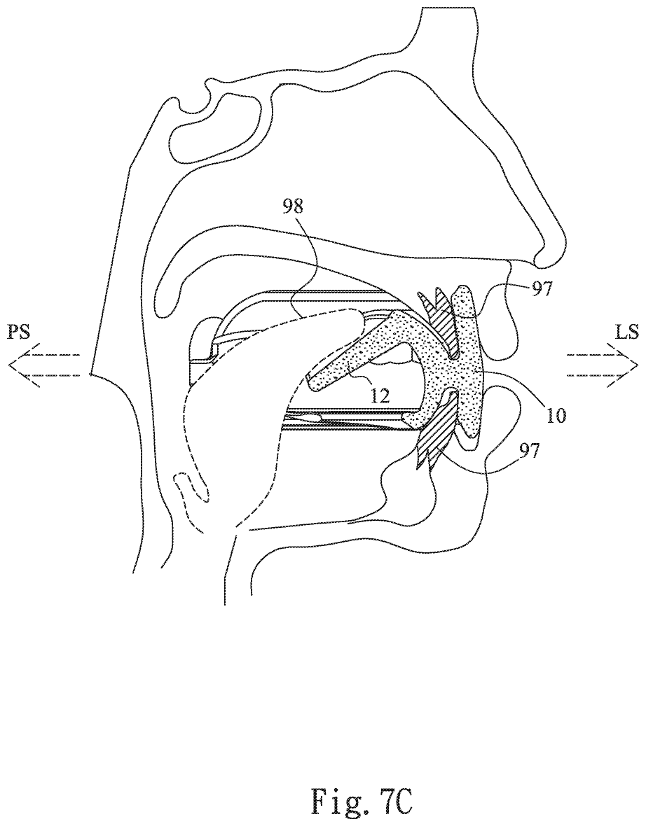

Please refer to FIG. 6A and FIG. 6B, FIG. 6A˜6B are schematic diagrams showing perspective view and top view of the first stage orthodontic appliance. As shown in FIG. 6A˜6B, physical model of the first stage orthodontic appliance 10 has main body, i.e. first stage arch 11, and a pluralities of first stage anchoring troughs C17 and first stage adapting troughs C18 disposed on the main body of the first stage orthodontic appliance 10. When orthodontic treatment is made, the first stage orthodontic appliance 10 can be bitten within patient's mouth as shown in FIG. 7A, and then aligned with the dentition 97 of upper jaw or lower jaw. Afterward, as shown in FIG. 7B and FIG. 7C, dental patient closes the mouth to have the upper jaw and lower jaw of dentition 97 approached to each other, and to bite the first stage orthodontic appliance 10.

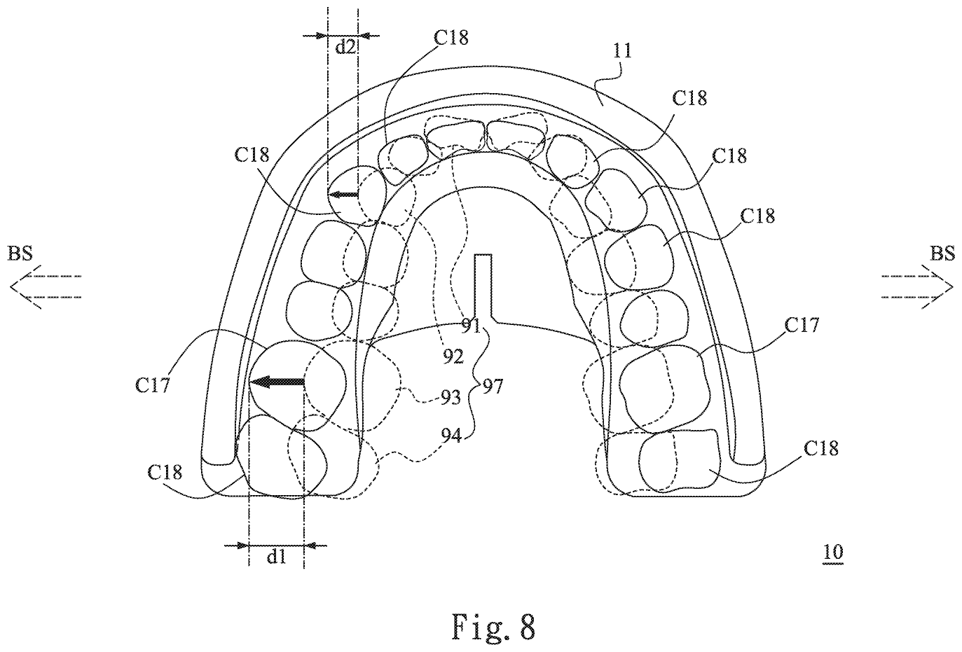

Further refer to FIG. 5A-5C and FIG. 8, in which FIG. 8 is schematic diagram showing structural relationship between patient's teeth and troughs of the first stage orthodontic appliance. When the first stage orthodontic appliance 10 is utilized within patient's mouth, the first stage anchoring troughs C17 are expected to match up with the first molars 93; therefore the first stage anchoring troughs C17 needs to be disposed at distal of the first expanding space d1 along Buccal Side direction BS. Then, the pluralities of first stage adapting troughs C18 are expected to match up with other teeth (including incisors 91, canines 92, other molars 94 but excluding first molars 93); therefore the first stage adapting troughs C18 need to be disposed at distal of the related second expanding spaces d2 along Buccal Side direction BS. In this manner, setting and adjustment of the first expanding space d1 are able to decide the spatial relationship (such as relative position, orientation or azimuth) between the first molar 93 and the first stage arch 11 when the Step 12 within FIG. 3 is made. Furthermore, setting and adjustment of the second expanding space d2 are able to decide the spatial relationship between the other teeth (including incisors 91, canines 92, other molars 94 but excluding first molars 93) and the first stage arch 11 when the Step 14 within FIG. 3 is made. Hence, the greater expanding space d1 and d2 are set, more push force will be generated to move the tooth. Thus, dentist or dental technician can designate future expecting movement or rotation of each teeth, and then arrange these movement and rotation on the first stage orthodontic appliance 10, so that the first stage orthodontic appliance 10 can have the structural/spatial information of teeth's movement and rotation. Clinically the present invention of the first stage orthodontic appliance 10 can have orthodontic function through pushing one tooth to move, or shifting several teeth to expand dental arch. Therefore dental patient can make his/her teeth move, rotate and thus meet the pre-defined dentition of the first stage orthodontic appliance 10, to implement first stage orthodontic treatment.

Besides as shown in FIG. 6A, the first stage orthodontic appliance 10 further has a first stage tongue support 12 disposed at inner side of the first stage arch 11. The first stage tongue support 12 has a slot 13 disposed on midline of the first stage arch 11. The slot 13 can be a crevasse or U-shaped groove, to accommodate human's ankyloglossia (also called tongue-tie). When dental patient bites the first stage orthodontic appliance 10, he/she can put the tongue above the first stage tongue support 12, to relieve the uncomfortable feeling of the patient and increase his/her intention for this orthodontic treatment. Additionally as shown in FIG. 7C, the first stage tongue support 12 gradually slopes downward along the opposite of Labial Side direction LS, namely the altitude of the first stage tongue support 12 gradually decrease along the Palate Side direction PS. When the dental patient bites the first stage orthodontic appliance 10, he/she can put his/her tongue 98 above the first stage tongue support 12, so as to raise the position of the tongue 98 and relax the throat muscles, and then open the respiratory tract to avoid human's airway obstruction. Besides, it can also have advantages such as reducing or eliminating symptom of “mouth breathing” which is caused by snoring and lowered tongue position. Therefore the first stage tongue support 12 disposed on the first stage orthodontic appliance 10 can have breath-training function for the patient who suffered sleep apnea or severe snoring, so as to reduce the snoring noise and frequency, and improve his/her sleep quality.

After the Step 11 to Step 17, as shown in FIG. 3, are implemented, it means that fabricating the first stage orthodontic appliance 10, as shown in Step X101 of FIG. 2, is carried out. Then and therefore the patient can have orthodontic treatment through the first stage orthodontic appliance 10 (Step X102 within FIG. 2). The purpose of first stage orthodontic treatment as presented in Step X102 is to enlarge and expand the size of patient's dentition 97, and afterward move and rotate each individual tooth within the dentition 97. Specifically, enlarging or expanding patient's dentition 97 needs to extend the dental arch toward the Buccal Side direction BS (i.e. toward left side and right side), so that the width of the dental arch in the Buccal Side directions BS is thus widened. Then, according to future size and space of patient's permanent teeth, the position and orientation of all teeth can be designated by the dentist or dental technician. The first stage orthodontic treatment may have functions of growing the alveolar bone among the locations which has insufficient space for tooth growth, or inhibit the growth of bone in the case of overgrowth of alveolar bone. Furthermore, it can not only increase the space for rotation and displacement of each individual tooth, but also make the facial and chin contour more beautiful. The clinical orthodontic result of the first stage orthodontic appliance 10 had demonstrated that the patient may have about 1 mm displacement within 2 to 6 weeks to move individual tooth of patient's dentition 97.

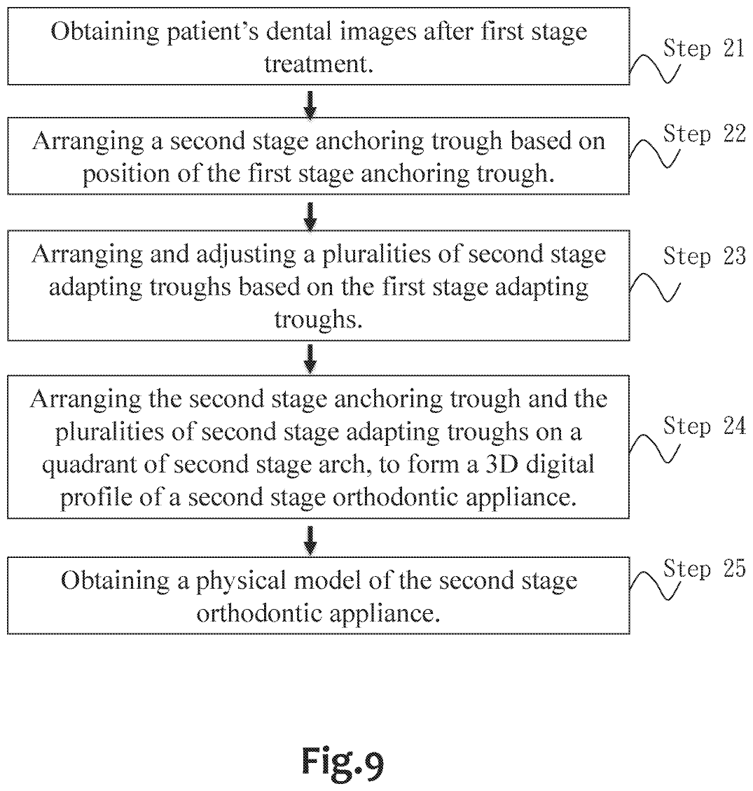

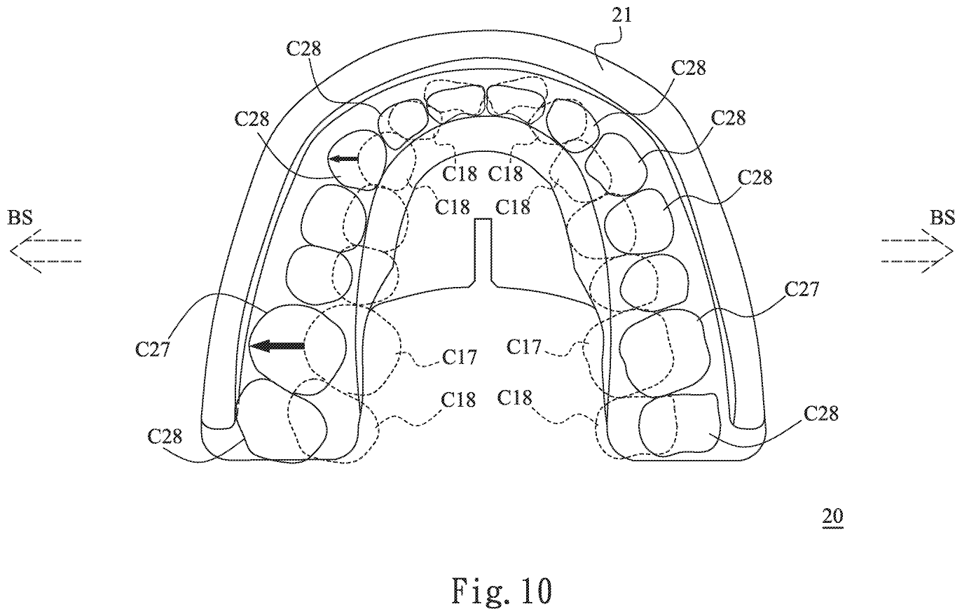

Please refer to FIG. 9 and FIG. 10, FIG. 9 is flow chart showing the manufacturing method of second stage orthodontic appliance; FIG. 10 is schematic diagram showing structural relationship between troughs of the first stage orthodontic appliance and the second stage orthodontic appliance. The Step X103, as shown in FIG. 2, can be further broken down into Step 21 to Step 25 as shown in FIG. 9. Firstly, the patient's dental images after first stage treatment can be obtained (Step 21). The way of obtaining dental images includes Computed Tomography, X-Ray, nuclear magnetic resonance or ultrasonic instruments, or oral scan or dental arch molding technique, etc., which is identical to the manufacturing method of the first stage orthodontic appliance 10. Next, a second stage anchoring trough C27 is arranged, based on position of the first stage anchoring trough C17 (Step 22), then a pluralities of second stage adapting troughs C28 are arranged and adjusted, based on position of the first stage adapting troughs C18 (Step 23). More specifically in Step 23, the second stage adapting troughs C28 can be disposed through shifting the sites of the first stage adapting troughs C18 by ¼ to ⅓ of tooth's section width, or rotating the sites of the first stage adapting troughs C18 by an angle less than 30 degree. Namely the exact position of the second stage adapting troughs C28 are set by limited displacement or rotating angle based on the first stage adapting troughs C18. In this reason, as shown in FIG. 10, dentist or dental technician can decide the pre-designated movement or rotation of each individual tooth for second stage orthodontic treatment, based on the position of first stage anchoring troughs C17 and first stage adapting troughs C18. Afterward the second stage anchoring trough C27 and the pluralities of second stage adapting troughs C28 on a quadrant of second stage arch 21 are able to be arranged, so as to form a 3D digital profile of a second stage orthodontic appliance 20 (Step 24). Thus a physical model of the second stage orthodontic appliance 20 is obtained (Step 25), such that the second stage anchoring troughs C27 disposed on the second stage orthodontic appliance 20 meet Class I occlusion relationship of Angle's Classification. By the way, the digital profile of troughs C27 and C28, physical model of the second stage orthodontic appliance 20, and the manufacturing method of present invention as described in Step 24 and Step 25 are similar to the first stage orthodontic appliance 10.

Thus, physical model of the second stage orthodontic appliance 20 has main body, i.e. second stage arch 21, and a pluralities of second stage anchoring troughs C27 and second stage adapting troughs C28 disposed on the main body of the second stage orthodontic appliance 20. Besides, the location and orientation of the second stage anchoring troughs C27 is identical to the first stage anchoring troughs C17, and the second stage adapting troughs C28 are set and arranged through shifting or rotating the first stage adapting troughs C18.

In this manner, the dental patient can have the second stage orthodontic treatment through the second stage orthodontic appliance 20 (Step X104 within FIG. 2). The main purposes of the second orthodontic treatment are addressed as followed; firstly, patient's first molars 93 can be met and located at the second stage anchoring troughs C27 of the second stage orthodontic appliance 20, for anchoring and positioning of occlusion. Secondly, utilizing the second stage anchoring troughs C 27 as anchorage to move or rotate each individual tooth through occlusion training, so that all teeth can be afterward moved and rotated to meet the pre-designated location (i.e. location of the second stage adapting troughs C28), and have function of occlusal adjustment and occlusal correction for patient's dentition 97. Therefore patient's dentition 97 may eventually meet Class I occlusion relationship of Angle's Classification, as well as move the upper and lower jaws to Centric Relation (CR), and thus improve the stability of patient's occlusion. Please noted that, the position of Centric Occlusion (CO) is the position where the upper teeth and lower teeth bite in the closest-fit condition, namely the upper teeth and lower teeth bite to meet the largest occlusal surface. Furthermore, the Centric Relation (CR) is the mandibular jaw position in which the head of the condyle is situated as far anterior and superior as it possibly can within the mandibular glenoid fossa, where CR is the most stable situation to all teeth. Normally, the most prefect situation is that the position of CO has 0.5 mm to 1 mm displacement away from the position of CR. The present invention of orthodontic suite may adjust and correct the jaws in multi stages, thus the patient who suffers malocclusion of Class II or Class III occlusion relationship of Angle's Classification can have orthodontic treatment, and then make the patient's first molars 93 be gradually moved and rotated to meet Class I occlusion relationship of Angle's Classification; in which the first stage anchoring troughs C17 and second stage anchoring troughs C27 are designated and set to meet Class I occlusion relationship of Angle's Classification. Therefore upper jaw and lower jaw correction through the present invention of orthodontic suite may have the first molars 93 moved and guided to be anchored at position to meet Class I occlusion relationship, and keep the upper jaw and lower jaw in the Centric Relation for the sake of dental health. If the dental patient is in the period of deciduous teeth falling off and permanent teeth germinating, e.g. around or before adolescence, then the orthodontic suite of the present invention can also adjust the contour of the patient's face and jaw, to let him/her have a more beautiful cheek shape. If the patient suffers from muscle dysfunction, having problems such as dysphagia with tongue prick or reverse swallowing, can improve his/her insufficiency of chewing or unilateral mastication through the occlusal training of the present invention. Since the 1990, it is found that there are dental bone forming cells (i.e. osteoblast) and bone resorption cells (i.e. osteoclast) in the alveolar bone, which affect the growth or contraction of the upper and lower jaw. When dental patient use the orthodontic suite of present invention, it can stimulate patient's oral sensitive cells through occlusion of upper jaw and lower jaw, and then convert the occlusal force into biological nerve signals of human body, so as to activate the “bone forming cells” or “bone resorption cells”; therefore alveolar bone growth or resorption is thus controllable. In this manner, the alveolar bone of human may react to the designated contour of first stage orthodontic appliance 10 and second stage orthodontic appliance 20, so as to activate osteoblast or osteoclast cells, and afterward grow or contract alveolar bone to shape the chin contour when dental patient have orthodontic treatment through the orthodontic suite of present invention. Thus, correction for jawbone, mandibular bone growth or chin shaping is achievable. Generally speaking, orthodontic treatment through the orthodontic suite of present invention can take advantage of tissue change biology to treat overdevelopment of dental arch or insufficient alveolar bone, and have functions of alveolar bone shaping, bone repair, bone correction and arranging teeth toward correct positions.

The second stage orthodontic appliance 20 may also have a second stage tongue support and a slot 13, so that the patient who bites the second stage orthodontic appliance 20 can raise altitude of his/her tongue 98, so as to relax the throat muscles and then open the respiratory tract to avoid human's airway obstruction. Additionally, it can also have advantages such as reducing or eliminating symptom of “mouth breathing” which is caused by snoring and lowered tongue position, and even have breath-training function for the patient who suffered sleep apnea or severe snoring, so as to reduce the snoring noise and frequency.

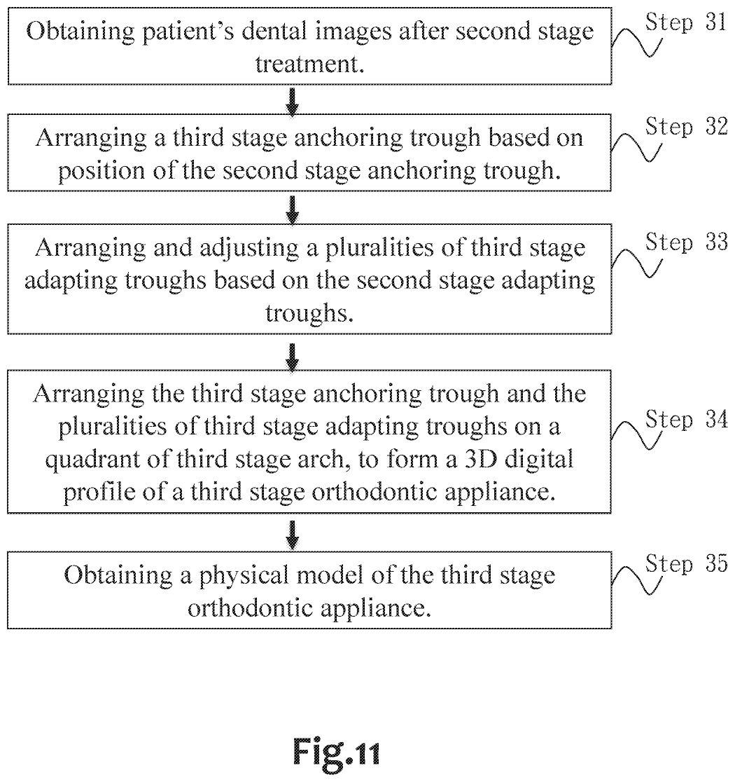

Next, dentist can decide whether a third stage orthodontic treatment is needed, according to clinic condition of the patient after the first stage and second stage treatment are made. If needed, the third stage orthodontic appliance is therefore required to be fabricated. Please refer to FIG. 11, FIG. 11 is flow chart showing the manufacturing method of third stage orthodontic appliance. As shown in FIG. 11, patient's dental images after second stage treatment are obtained (Step 31), then a third stage anchoring trough based on position of the second stage anchoring trough C27 is arranged (Step 32). Afterward a pluralities of third stage adapting troughs based on the second stage adapting troughs C28 are arranged and adjusted (Step 33), and eventually the third stage anchoring trough and the pluralities of third stage adapting troughs are arranged on a quadrant of third stage arch, so as to form a 3D digital profile of a third stage orthodontic appliance (Step 34). Then, a physical model of the third stage orthodontic appliance is obtained (Step 35). Therefore, the third stage orthodontic appliance comprises a third stage arch, and the third stage arch has pluralities of third stage anchoring troughs and third stage adapting troughs; such that positions of the third stage anchoring troughs are identical to positions of the second stage anchoring troughs C27; the positions of the third stage adapting troughs are arranged through shifting the positions of the second stage adapting troughs C28 by ¼ to ⅓ of tooth's section width, or rotating the sites of the second stage adapting troughs C28 by an angle less than 30 degree. Descriptively, the fabricating method of the third stage orthodontic appliance is similar to the second stage orthodontic appliance 20, thus further address is not repeated. The clinic purpose of the third stage orthodontic treatment is a supplement or follow-up to the second stage treatment, so as to have greater correction and adjustment orthodontic effect.

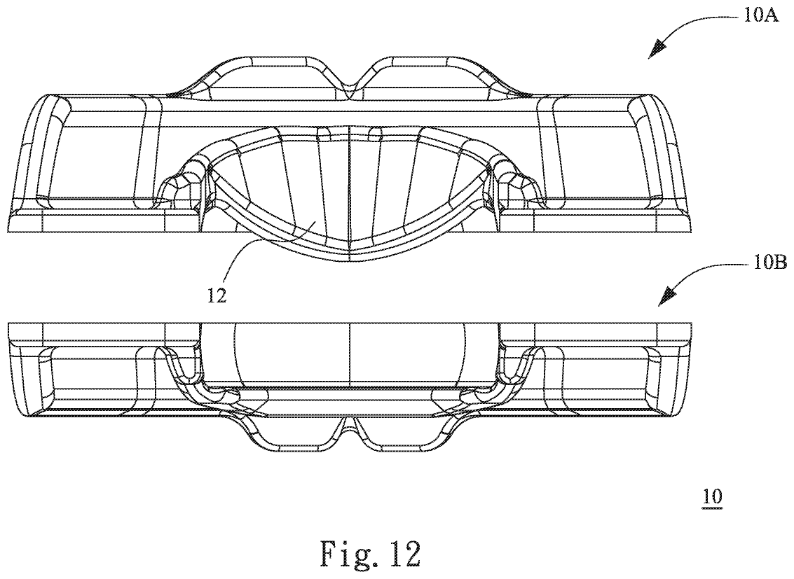

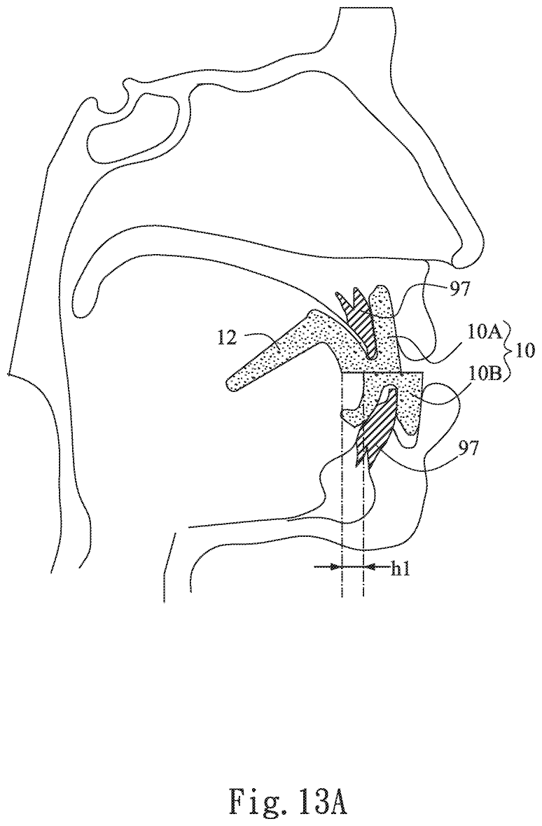

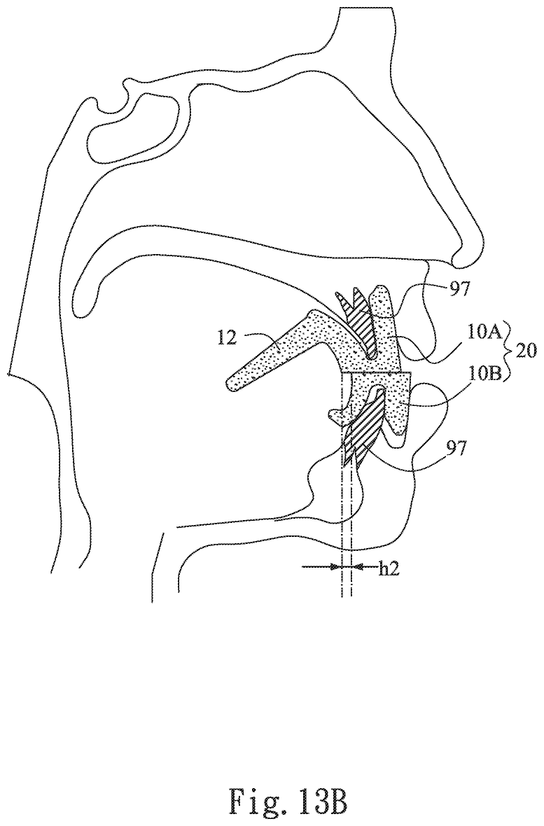

Please refer to FIG. 12, FIG. 12 is schematic diagram showing the orthodontic appliance with the higher part arch and the under part arch separately fabricated. As shown in FIG. 12, the first stage orthodontic appliance 10 can be separately fabricated, thus the first stage orthodontic appliance 10 has a higher part arch 10A and a under part arch 10B respectively. The higher part arch 10A can match dental patient's upper jaw and meet the profile of the upper jaw; the under part arch 10B can match dental patient's lower jaw and meet the profile of the lower jaw. After physical model of the first stage orthodontic appliance 10 is manufactured (Step 17 within FIG. 3), then the higher part arch 10A and the under part arch 10B can be adhered and combined. The combined first stage orthodontic appliance 10 can be utilized in the first stage orthodontic treatment as shown in FIG. 13A. When upper jaw and lower jaw of dentition 97 has greater malocclusion, the higher part arch 10A and the under part arch 10B can have a greater shift h1 to adhere together, so that the first stage orthodontic appliance 10 can treat patient's greater malocclusion. After the first stage orthodontic treatment is finished, the malocclusion between upper jaw and lower jaw is reduced; in this circumstance, it enters the second stage orthodontic treatment as shown in FIG. 13B, the higher part arch 10A and the under part arch 10B is adhered and combined with smaller shift h2, so as to form the second stage orthodontic appliance 20. Namely the first stage orthodontic appliance 10 has greater shift h1 between the higher part arch 10A and the under part arch 10B, and the second orthodontic appliance 20 has smaller shift h2. In this manner, dental technician can design the profile of the orthodontic appliance once, and then fabricate and obtain the physical model of orthodontic appliances for multi stages (including first, second and third stage) through arranging different shift h1 or h2. Therefore laboring cost on profile design of the orthodontic appliance is reduced.

Summarily, the method of orthodontic suite can fabricate a multistage orthodontic appliance 10, 20, to pre-designate a shifting or rotating position for patient's malocclusive, malposition or abnormal aligned teeth and jaws, and even reshape and regenerate jawbone through biological morphology of orthodontics, and further address the hypoplasia or overdevelopment of dental arch. Besides, the first stage tongue support 12 or second stage tongue support 22 disposed can raise the position of the tongue 98 and relax the throat muscles, and then open the respiratory tract to avoid human's airway obstruction. It can further have advantages such as reducing or eliminating symptom of “mouth breathing” which is caused by snoring and lowered tongue position; therefore the multi stage orthodontic appliance 10 and 20 can have breath-training function for the patient who suffered sleep apnea or severe snoring, so as to reduce the snoring noise and frequency, and improve his/her sleep quality. Moreover, the orthodontic suite of present invention does not use metal archwire of traditional means to correct teeth, so that the issues of positioning or controllability of movement or orientation for teeth are eventually reduced, and thus higher accuracy of correcting azimuth and movement are achievable. Therefore orthodontics including teeth alignment, jaws correction, teeth reshape, reposition, and occlusion arrangement, etc., are able to treated through the present invention, and patient can have tooth brushing and cleaning with ease and convenience as usual during the orthodontic suite is utilized. So adaptability of the orthodontic suite of present invention to all ages including adults and kids is made, thus it may have huge clinic practicability and commercial advantages.

The figures and descriptions supra set forth illustrated the preferred embodiments of the instant disclosure; however, the characteristics of the instant disclosure are by no means restricted thereto. All changes, alternations, combinations or modifications conveniently considered by those skilled in the art are deemed to be encompassed within the scope of the instant disclosure delineated by the following claims.

The figures and descriptions supra set forth illustrated the preferred embodiments of the instant disclosure; however, the characteristics of the instant disclosure are by no means restricted thereto. All changes, alternations, combinations or modifications conveniently considered by those skilled in the art are deemed to be encompassed within the scope of the instant disclosure delineated by the following claims.

What is claimed is:

1. A orthodontic suite, for multistage treating malocclusion or abnormal alignment of the teeth and jaws, comprising:
a first stage orthodontic appliance (10), having a first stage arch (11), a pluralities of first stage anchoring troughs (C17) and a pluralities of first stage adapting troughs (C18), with the first stage anchoring troughs (C17) and the first stage adapting troughs (C18) disposed on the first stage arch (11), the first stage anchoring troughs (C17) being disposed through shifting sites of patient's first molars (93) along Buccal Side direction (BS) by a first expanding space (d1), the first stage adapting troughs (C18) being disposed through shifting sites of patient's other teeth along Buccal Side direction (BS) by a second expanding space (d2); and
a second stage orthodontic appliance (20), having a second stage arch (21), a pluralities of second stage anchoring troughs (C27) and a pluralities of second stage adapting troughs (C28), with the second stage anchoring troughs (C27) and the second stage adapting troughs (C28) disposed on the second stage arch (21), the second stage anchoring troughs (C27) being disposed at the same sites with the first stage anchoring troughs (C17), the second stage adapting troughs (C28) being disposed through shifting or rotating sites of the first stage adapting troughs (C18).

2. The orthodontic suite according to claim 1, wherein the first stage anchoring troughs (C17) or second stage anchoring troughs (C27) meet Class I occlusion relationship of Angle's Classification.

3. The orthodontic suite according to claim 1, wherein the second expanding space (d2) is smaller than or equal to 1.2 times the first expanding space (d1), and larger than 0.2 times the first expanding space (d1).

4. The orthodontic suite according to claim 1, wherein the second stage adapting troughs (C28) are disposed through shifting the sites of the first stage adapting troughs (C18) by one fourth to one third of tooth's section width, or rotating the sites of the first stage adapting troughs (C18) by an angle less than 30 degree.

5. The orthodontic suite according to claim 1, further having a third stage orthodontic appliance, wherein the third stage orthodontic appliance has a third stage arch, a pluralities of third stage anchoring troughs and a pluralities of third stage adapting troughs, with the third stage anchoring troughs and the third stage adapting troughs disposed on the third stage arch; the third stage anchoring troughs being disposed at the same sites with the second stage anchoring troughs (C27), the third stage adapting troughs being disposed through shifting or rotating sites of the second stage adapting troughs (C28).

6. The orthodontic suite according to claim 5, wherein the third stage adapting troughs are disposed through shifting the sites of the second stage adapting troughs (C28) by one fourth to one third of tooth's section width, or rotating the sites of the second stage adapting troughs (C28) by an angle less than 30 degree.

7. The orthodontic suite according to claim 1, further having a first stage tongue support (12) and a second stage tongue support (22), with the first stage tongue support (12) disposed at inner side of the first stage arch (11) and the second stage tongue support (22) disposed at inner side of the second stage arch (21).

8. The orthodontic suite according to claim 7, wherein the first stage tongue support (12) or the second stage tongue support (22) gradually slopes downward along the opposite of Labial Side direction (LS).

9. The orthodontic suite according to claim 7, wherein the first stage tongue support (12) has a slot (13) disposed on midline of the first stage arch (11), or the second stage tongue support (22) has a slot (13) disposed on midline of the second stage arch (21).

10. A manufacturing method of orthodontic suite, comprising:
Step 11: obtaining patient's dental images;
Step 12: setting a first expanding space (d1) from the patient's first molar (93) toward patient's cheek along Buccal Side direction (BS);
Step 13: arranging a first stage anchoring trough (C17) through shifting position of the first molar (93) by the first expanding space (d1);
Step 14: setting a pluralities of second expanding spaces (d2) on other teeth toward the Buccal Side direction (BS) based on relative positions with the first stage anchoring trough (C17);
Step 15: arranging a pluralities of first stage adapting troughs (C18) on the distal of the second expanding spaces (d2) according to category of the teeth;
Step 16: arranging the first stage anchoring trough (C17) and the pluralities of first stage adapting troughs (C18) on a quadrant of first stage arch (11) to form a 3D digital profile of a first stage orthodontic appliance (10);
Step 17: obtaining a physical model of the first stage orthodontic appliance (10);
Step 21: obtaining patient's dental images after first stage treatment;
Step 22: arranging a second stage anchoring trough (C27) based on position of the first stage anchoring trough (C17);
Step 23: arranging and adjusting a pluralities of second stage adapting troughs (C28) based on the first stage adapting troughs (C18);
Step 24: arranging the second stage anchoring trough (C27) and the pluralities of second stage adapting troughs (C28) on a quadrant of second stage arch (21), to form a 3D digital profile of a second stage orthodontic appliance (20);
Step 25: obtaining a physical model of the second stage orthodontic appliance (20).

11. The manufacturing method of orthodontic suite according to claim 10, wherein the first stage anchoring trough (C17) or second stage anchoring trough (C27) meets Class I occlusion relationship of Angle's Classification.

12. The manufacturing method of orthodontic suite according to claim 10, wherein the second expanding space (d2) of patient's incisor (91) in Step 14 is smaller than or equal to 0.4 times the first expanding space (d1), and larger than 0.2 times the first expanding space (d1).

13. The manufacturing method of orthodontic suite according to claim 10, wherein the second expanding space (d2) of patient's canine (92) in Step 14 is smaller than or equal to 0.8 times the first expanding space (d1), and larger than 0.4 times the first expanding space (d1).

14. The manufacturing method of orthodontic suite according to claim 10, wherein the second expanding space (d2) of patient's other molars (94) in Step 14 are smaller than or equal to 1.2 times the first expanding space (d1), and larger than 0.8 times the first expanding space (d1).

15. The manufacturing method of orthodontic suite according to claim 10, wherein the second stage adapting troughs (C28) in the Step 23 are arranged through shifting the positions of the first stage adapting troughs (C18) by one fourth to one third of tooth's section width.

16. The manufacturing method of orthodontic suite according to claim 10, wherein the second stage adapting troughs (C28) in the Step 23 are arranged through rotating the sites of the first stage adapting troughs (C18) by an angle less than 30 degree.

17. The manufacturing method of orthodontic suite according to claim 10, wherein the first stage orthodontic appliance (10) or the second stage orthodontic appliance (20) is composed of a higher part arch (10A) and a under part arch (10B), with the higher part arch (10A) and the under part arch (10B) separately fabricated.

18. The manufacturing method of orthodontic suite according to claim 10, further comprising:
Step 31: obtaining patient's dental images after second stage treatment;
Step 32: arranging a third stage anchoring trough based on position of the second stage anchoring trough (C27);
Step 33: arranging and adjusting a pluralities of third stage adapting troughs based on the second stage adapting troughs (C28);
Step 34: arranging the third stage anchoring trough and the pluralities of third stage adapting troughs on a quadrant of third stage arch, to form a 3D digital profile of a third stage orthodontic appliance;
Step 35: obtaining a physical model of the third stage orthodontic appliance.

19. The manufacturing method of orthodontic suite according to claim 18, wherein the third stage orthodontic appliance is composed of a higher part arch (10A) and a under part arch (10B), with the higher part arch (10A) and the under part arch (10B) separately fabricated.
